(19) United States
(12) Patent Application Publication
(10) Pub. No.: US 2020/0155361 A1
(43) Pub. Date: May 21, 2020

(54) EXTENSIBLE DRESSINGS

(71) Applicants: KCI USA, INC., San Antonio, TX (US); SYSTAGENIX WOUND MANAGEMENT, LIMITED, West Sussex (GB)

(72) Inventors: William PIGG, Elvington (GB); Sally STEPHENS, Skipton (GB)

(21) Appl. No.: 16/615,339

(22) PCT Filed: May 21, 2018
(86) PCT No.: PCT/US2018/033636

§ 371 Date: Nov. 20, 2019

Related U.S. Application Data
(60) Provisional application No. 62/509,594, filed on May 22, 2017.

(51) Int. Cl. A61F 13/02; A61L 15/58; A61F 13/00

(52) U.S. Cl. CPC A61F 13/0289; A61L 15/58; A61F 13/0213; A61F 13/00029; A61L 2300/404; A61F 13/00042; A61F 13/00012; A61L 2300/414; A61F 2013/00174; A61F 13/00038

(57) ABSTRACT

Described herein are dressings for incision management comprising a dressing layer that comprises: an elastic layer such as a hydrocolloid or foam layer; and an absorbent layer coupled, for example by lamination, to the elastic layer and which comprises: (i) from about 45% to about 90% carboxymethyl cellulose fibers; and (ii) from about 10% to about 55% elastic reinforcing fibers. Though the elastic layer may be substantially elastically deformable under tissue treatment conditions and the absorbent layer may be not elastically deformable under tissue treatment conditions, the coupling together of the elastic layer and the absorbent layer may result in a dressing layer, and advantageously a dressing, that is substantially elastically deformable under tissue treatment conditions. Methods for manufacturing such dressings and of eliminating, minimizing, or reducing edema using such dressings are also described herein.

### CROSS-REFERENCE TO RELATED APPLICATIONS

This application claims the benefit of priority to U.S. Provisional Application No. 62/509,594, filed on May 22, 2017, which is incorporated herein by reference in its entirety.

### TECHNICAL FIELD

The subject matter set forth in the appended claims relates generally to treating tissue, including extensible dressings having an absorbent layer and elastic fibers.

### BACKGROUND

A wide variety of materials and devices, generally characterized as “wound dressings,” are generally known in the art for use in treating an injury or other disruption of tissue. Such wounds may be the result of trauma, surgery, or disease, and may affect skin or other tissues. In general, wound dressings may control bleeding, absorb wound exudate, ease pain, assist in debriding the wound, protect wound tissue from infection, or otherwise promote healing and protect the wound from further damage.

Although the clinical benefits and advantages of wound dressings may be widely accepted, improvements to wound dressings may benefit healthcare providers and patients.

### BRIEF SUMMARY

New and useful compositions of dressing layers and dressings including such a dressing layer, methods for manufacturing same, and methods for using the same are set forth in the appended claims. Illustrative embodiments are also provided to enable a person skilled in the art to make and use the claimed subject matter.

Some examples may comprise a dressing containing a dressing layer that comprises an elastic layer, such as a hydrocolloid or foam layer, and an absorbent layer coupled to the elastic layer. The absorbent layer may comprise from about 45% to about 90% carboxymethyl cellulose fibers and from about 10% to about 55% elastic reinforcing fibers. In some embodiments, the elastic layer may be substantially elastically deformable under tissue treatment conditions, while the absorbent layer may be not elastically deformable under tissue treatment conditions. Nevertheless, the elastic layer and the absorbent layer, when coupled together to form the dressing layer, may be substantially elastically deformable under tissue treatment conditions, as may the entire dressing.

Another example relates to a method of manufacturing a dressing having an absorbent layer with elastic reinforcing fibers. In some embodiments, an elastic layer such as a hydrocolloid or foam layer may be extended to a length at least 50% greater than an original length of the elastic layer. An absorbent layer, which in some embodiments may comprise from about 45% to about 90% carboxymethyl cellulose fibers and from about 10% to about 55% non-gelling fibers, may be laminated onto the extended elastic layer, thereby forming a composite dressing layer. In some embodiments, the lamination can occur using a layer comprising an adhesive, so as to couple the elastic layer to the layer comprising the adhesive and to couple the layer comprising the adhesive to the absorbent layer. In such embodiments, the layer comprising the adhesive can be deposited on the elastic layer, whether before, during, or after the extension of the elastic layer, to enable the coupling steps. In the forming of the composite dressing layer, only a portion of the fibers in the absorbent layer may be coupled to the extended elastic layer, for example to allow for adherence during relaxation. Thereafter, the composite laminate may be relaxed to return the elastic layer to its original length. This relaxation step may advantageously cause bunching of the portion of the fibers in the absorbent layer that are not coupled to the elastic layer, while allowing the portion of the fibers in the absorbent layer that are coupled, albeit indirectly, to retain their coupling and advantageously to transfer some elastic character to the absorbent layer. In particular embodiments, the portion of the fibers in the absorbent layer coupled to the extended elastic layer may be sufficient for the composite laminate, the dressing, or both to exhibit a substantially elastic recovery under tissue treatment conditions. In some embodiments, the layer comprising the adhesive may include non-gelling fibers, and the coupling of the elastic layer to the layer comprising the adhesive and to couple the layer comprising the adhesive to the absorbent layer may occur approximately simultaneously, for instance by heating the non-gelling fibers to a temperature of at least about 10° C. below a melting point of the non-gelling fibers for about 1 minute to about 16 hours to establish effective coupling.

Another example relates to a method of eliminating, minimizing, or reducing edema for a wound site surrounded by tissue. The method may comprise positioning a dressing described herein or made according to a method described herein near the wound site and releasably adhering the dressing to the tissue.

Objectives, advantages, and one or more preferred modes of making and using the claimed subject matter may be understood by reference to the accompanying drawings, in conjunction with the following detailed description of illustrative embodiments.

### DRAWINGS

FIG. 1 is a perspective view in cross-section of a dressing according to this specification.

FIG. 2 is a diagram of the dressing of FIG. 1 with a therapy system.

It should be noted that the figures set forth herein are intended to exemplify the general characteristics of materials and methods among those of the present invention, for the purpose of the description of certain embodiments. The figures may not precisely reflect all the characteristics of any given embodiment, and are not necessarily intended to define or limit specific embodiments within the scope of this invention.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

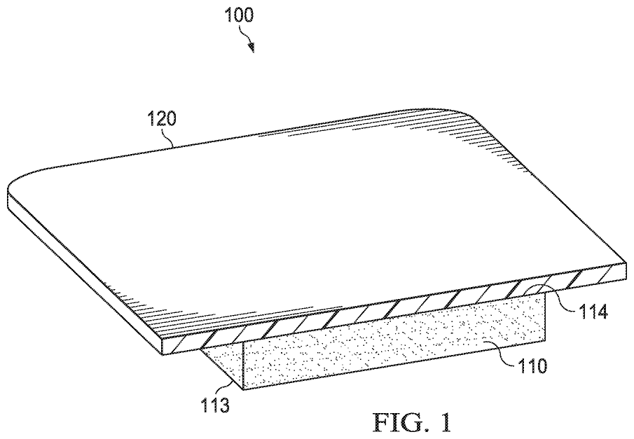

Disclosed herein are embodiments of a dressing layer, embodiments of composite dressings including such a dressing layer, and embodiments of therapy systems including same. Also disclosed herein are embodiments of related methods, such as methods of making and methods of using the disclosed dressing layers, dressings, and therapy systems. For example, FIG. 1 illustrates an embodiment of a dressing 100 suitable for treating a wound.

As used herein, “tissue site” is intended to broadly refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), skin flaps, and grafts, for example. The term “tissue site” may also refer to areas of any tissue that are not necessarily wounded or defective, but are instead areas in which it may be desirable to add or promote the growth of additional tissue, such as granulation. As noted above, the tissue site can refer to an area of tissue where prophylactic treatment is desired, even without a wound or defect, for example with respect to pressure ulcer prevention.

In some embodiments, a dressing may include one or more dressing layers configured to interface with a tissue site. For example, in the embodiment of FIG. 1, the dressing 100 may include a dressing layer 110. The dressing layer 110 may generally be configured to be positioned on, over, in, adjacent to, or otherwise in contact with (collectively, “near”) a tissue site.

### Dressing Layer

In various embodiments, the dressing layer 110 may be configured so as to be in contact with a portion of the tissue site, substantially all of the tissue site, or the tissue site in its entirety. If the tissue site is a wound, for example, the dressing layer 110 may partially or completely fill the wound, or may be placed over or near the wound. In various embodiments, the dressing layer 110 may take many forms, and may have many sizes, shapes, or thicknesses depending on a variety of factors, such as the type of treatment being implemented or the nature and size of a tissue site. For example, the size and shape of the dressing layer 110 may be adapted to the contours of deep and irregular shaped tissue sites, may be configured so as to be adaptable to a given shape or contour, or both. Moreover, in some embodiments, any or all of the surfaces of the dressing layer 110 may comprise projections or an uneven, course, or jagged profile that can, for example, induce strains and stresses on a tissue site, which may be effective to promote granulation at the tissue site.

In some embodiments, the dressing layer 110 may be in substantially sheet form. For example, the dressing layer 110 may comprise a generally planar structure having two opposite-facing planar surfaces and a depth or thickness orthogonal to the planar surfaces. More particularly, for example, the dressing layer 110 may comprise a first surface 113 opposite a second surface 114. The first surface 113 may be adapted to contact a tissue site, having a surface area sufficient to cover an appropriate portion, if not all, of the tissue site. For example, a surface area from about 1 cm² to about 4000 cm² may be suitable for many applications. In various embodiments, the first surface 113 and the second surface 114 may have any suitable shape, examples of which include, but are not limited to, triangles, squares, rhombuses, rhomboids, diamonds, rectangles, trapezoids, ellipses, ellipsoids, circles, semi-circles, pie-wedges, ovals, and various polygons having four, five, six, seven, eight, or more sides. These shapes may additionally or alternatively be adaptations of such common shapes. In some embodiments, shapes with typically rounded edges may be altered to be flatter, such as a rounded hexagonal/octagonal shape made by flattening the rounded edges of a circle. Additionally or alternatively, shapes with typically rounded edges may be altered to be sharper, such as a tear-drop shape made by sharpening a rounded end of an ellipse or ellipsoid, or such as an eye shape made by sharpening two rounded, opposing ends of an ellipse or ellipsoid. Further additionally or alternatively, shapes with typically pointed edges may be altered to be more rounded, such as for a blunt-ended triangle. Still further additionally or alternatively, shapes with typically flat edges may be altered to be more rounded, such as by converting the flat sides of any regular polygon to a sinusoidal edge to form a doily shape with an undulating, curvy edge. The shape and area of the back surface 114 may be customized to the location and type of tissue site onto which the dressing 100 is to be applied.

### Dressing Layer Composition

There can be various embodiments of the composition of the dressing layer. In some embodiments, the dressing layer 110 may be a single layer, whereas, in some embodiments, the dressing layer 110 may represent a multi-layer composite structure. For example, the dressing layer 110 may comprise at least two layers coupled to each other.

Layers of a multi-layer composite structure may be coupled to each other using any appropriate technique. In some embodiments, a lamination process can be used to couple layers together, particularly where neither of the layers to be coupled are fibrous or only one of the two layers is fibrous. In some embodiments, an adhesive can be used to directly or indirectly couple layers together. In such embodiments, direct adhesion can be used particularly where neither of the layers to be coupled are fibrous, and indirect adhesion can be used particularly where at least one of the two layers is fibrous.

In some embodiments where at least one of the layers is fibrous, a needling apparatus, such as a needle loom machine, can be used to couple the fibrous layer to the other layer, whether fibrous or porous. Needling apparatuses may be used to knit together different types of fibrous materials, such as non-wovens. Typically, in needling apparatuses, a bottom layer can be coupled to a top layer by co-feeding the two layers through the apparatus to be simultaneously needled. By operation of such apparatuses, one or more barbed needles can be punctured into/through the fibrous material(s). Differences in strength or connectivity in coupling can arise in needling treatments from different numbers or arrays of barbed needles, from different barbed needle puncture speeds or frequencies, from different sizes of barbs or needles, from the like, or from some combination thereof. Without being bound by theory, it is believed that the repeated action of the barbed needles may effectively entangle or interweave the fibers of one or the layers with either the fibers or pore structure of the other layer, resulting in effective coupling. Furthermore, more than two layers can be effectively coupled by needling. For example, in a first step, coupling can be attained by co-feeding a top layer and a bottom layer through a needling apparatus to form a multi-layer composition. Then, by controlling the depth of needling, the top layer of the multi-layer composite, which represents the lower “layer” in this second step, can be coupled to a (fibrous) upper layer by co-feeding both to the needling apparatus and by ensuring that the needling depth does not exceed the thickness of the top layer of the composite, thereby forming a three-layer composite formed by two couplings via the needling apparatus. This process can be repeated with any desired number of layers to attain more highly layered composites. Additionally or alternatively, it is possible in multi-layered composites having at least two couplings for at least one of the couplings can be accomplished via needling, while at least one of the other couplings can be accomplished via a different coupling method, such as via lamination.

In some embodiments, the dressing layer 110 can be configured to exhibit substantially elastic recovery under tissue treatment conditions. In various embodiments, however, at least one of the component layers of the dressing 100 may not exhibit substantially elastic recovery under tissue treatment conditions. For instance, in some embodiments, the dressing layer 110 may include a composite island structure, which may comprise an elastic layer, such as a foam layer, silicone layer, hydrocolloid layer, or an extensible non-woven layer, coupled to an absorbent layer that is not elastic. In this way, the elastic layer can enable the composite island, and thus the dressing 100, to exhibit substantially elastic recovery at tissue treatment conditions, which may be a desirable characteristic in combination with an ability to absorb generous amounts of liquid, such as saline or exudate. This may be particularly important when the ability to absorb liquid comes mainly from a layer that does not exhibit substantial elastic recovery.

In this context, a material that exhibits substantially elastic recovery under tissue treatment conditions is termed “elastic”, and a material that does not exhibit substantially elastic recovery under tissue treatment conditions is termed “not elastic”. In some embodiments, the dressing 100 or any layer(s) thereof such as the dressing layer 110, having a width and a length perpendicular to the width, may be considered elastic if it exhibits at most about 10%, advantageously about 5% or less or about 2% or less, permanent deformation when subjected to about 50% strain, relative to the length, for about 3 days at that strain level, using an Instron™ mechanical testing machine, for example, at an initial imposed strain rate of about 1% elongation per second up to the total strain value, at which point it can be held for the total strain time. Additionally or alternatively, in some embodiments, the dressing 100 or any layer(s) thereof such as the dressing layer 110, having a width and a length perpendicular to the width, may be considered elastic if it exhibits at most about 5%, advantageously about 2% or less or about 1% or less, permanent deformation when subjected to about 25% strain, relative to the length, for about 24 hours at that strain level, using an Instron™ mechanical testing machine, for example, at an initial imposed strain rate of about 10% elongation per minute up to the total strain value, at which point it can be held for the total strain time. Additionally or alternatively, in some embodiments, the dressing 100 or any layer(s) thereof such as the dressing layer 110, having a width and a length perpendicular to the width, may be considered elastic if it exhibits at most about 1%, and advantageously about 0%, permanent deformation when subjected to about 10% strain, relative to the length, for about 10 minutes at that strain level, using an Instron™ mechanical testing machine, for example, at an initial imposed strain rate of about 1% elongation per minute up to the total strain value, at which point it can be held for the total strain time. Instron™ testing may be performed at room temperature, such as ˜20-25° C., and at low relative humidity, for example ˜40% RH or less. Even though particular values are specified with respect to the strain test, materials, layers, or compositions may be considered elastic if optionally tested with one or more parametric deviations, including but not limited to: being conducted at a greater strain than specified (for example, between about 50% strain and about 100% strain, between about 25% strain and about 75% strain, or between about 10% strain and about 50% strain), relative to the length; being conducted for a longer time than specified (for example, between about 10 minutes and about 120 hours, between about 24 hours and about 96 hours, or between about 3 days and about 7 days) at the total strain level; and being conducted at an initial strain rate greater specified (for example, between about 1% elongation per minute and about 600% elongation per minute, between about 10% elongation per minute and about 1200% elongation per minute, or between about 1% elongation per second and about 40% elongation per second).

Substantial elastic recovery may be particularly important for treating tissue in areas of relatively high articulation or flexure, such as proximal to shoulder, elbow, knee, ankle, or hip joints (particularly knee or elbow joints). It may be desirable for the dressing 100 to be able to undergo significant local flexure and substantially retain its shape, and its contact with the tissue site, upon significant articulation. Maintaining shape or tissue contact can reduce the frequency of necessitated dressing changes, reduce tissue blistering from improper contact, increase exudate absorption, reduce healing time, increase patient comfort, or combinations thereof.

An absorbent layer is typically present in the dressing layer 110 as one or more of at least two layers. For example, the dressing layer 110 may comprise a composite island having one or more absorbent layers. The absorbent layer may comprise a non-woven material of predominantly non-woven fibers, in some embodiments. For example, the dressing layer 110 may comprise a composite island having one or more absorbent layers. The absorbent layer may comprise a non-woven material of predominantly non-woven fibers, in some embodiments. For example, in various embodiments, the absorbent layer may comprise from about 45 parts to about 95 parts by weight of cellulosic (for example, cellulose ether) fibers and from about 5 parts to about 55 parts by weight of reinforcing (for example, elastic reinforcing) fibers. In particular embodiments, the absorbent layer may comprise from about 50 parts to about 95 parts by weight, from about 45 parts to about 90 parts by weight, from about 50 parts to about 90 parts by weight, from about 60 parts to about 90 parts by weight, from about 65 parts to about 85 parts by weight, or from about 70 parts to about 90 parts by weight of cellulosic fibers and from about 50 parts to about 5 parts by weight, from about 55 parts to about 10 parts by weight, from about 50 parts to about 10 parts by weight, from about 45 parts to about 10 parts by weight, from about 40 parts to about 10 parts by weight, from about 35 parts to about 15 parts by weight, from about 30 parts to about 10 parts by weight, from about 30 parts to about 15 parts by weight, or from about 25 parts to about 10 parts by weight of reinforcing fibers. In some optional embodiments, biodegradable components may additionally be present in the absorbent layer, for example in amounts from about 1 part to about 20 parts by weight, such as from about 1 part to about 15 parts by weight or from about 1 part to about 10 parts by weight.

In some multi-layer embodiments, a surface of the absorbent layer not coupled to elastic layer can be oriented to be a lower layer, whereas, in other multi-layer embodiments, a surface of the elastic layer, for example the hydrocolloid or foam layer, not coupled to the absorbent layer can be oriented to be a lower or contact layer. In some embodiments, it may be advantageous for the absorbent layer to be separated from a tissue site, for example, such that the surface of the elastic layer not coupled to the absorbent layer can be oriented to be a lower or contact layer.

In some embodiments, the elastic layer is adherent and can extend over the absorbent layer and over at least a portion of the backing layer margin, such as substantially the entire backing layer margin. In such embodiments, the portion of the elastic layer extending over the backing layer margin may be coupled to the backing layer. In some embodiments, the elastic layer is non-adherent, and an adherent layer can be disposed over the backing layer margin and over at least a portion of a margin of the composite island, in order to enable the dressing to be adhered to a tissue site. In some embodiments, the elastic layer is non-adherent, and an adherent layer can be disposed over the entire backing layer, thereby adhering the composite island to the backing layer and covering the backing layer margin as well, in order to enable the dressing to be adhered to a tissue site.

If cellulosic fibers are present in the absorbent layer, the cellulosic fibers may be composed of at least one of carboxymethyl cellulose (CMC), carboxylethyl cellulose, hydroxyethyl cellulose, hydroxypropyl cellulose, hydroxypropylmethyl cellulose, and cellulose ethyl sulphonate (CES) (particularly carboxymethyl cellulose), for example. In some embodiments, the cellulosic component may be at least partially in a salt form, for example, comprising a physiologically acceptable cation such as sodium. CMC is commercially available from a variety of sources, such as under the tradenames Walocel™ (sold by The Dow Chemical Company) and Cekol® (sold by CP Kelco). If reinforcing fibers are present in the absorbent layer, for example, the reinforcing fibers may be composed of at least one of a polyurethane gel, an amide polymer such as Nylon 6,6, an olefin polymer such as HDPE, an ester polymer such as PET, and a modified acrylamide polymer. If biodegradable components are present in the absorbent layer, for example, the biodegradable components may be composed of, but not limited to, an alginic acid, an alginate salt, chitosan, chitin, a guar gum, a locust bean gum, a xanthan gum, a karaya gum, gelatin, pectin, a starch derivative, a glycosaminoglycan, a galactomannan, a chondroitin salt, heparin, a heparin salt, collagen, oxidized regenerated cellulose (ORC), hyaluronic acid, a hyaluronate salt, or a combination thereof. For such listed salt components, the salt components may include any reasonable counterions, such as sodium, calcium, ammonium, or the like, or combinations thereof. The biodegradable component(s) can be, for example, in the form of a film or a foam, such as an open-cell foam, a reticulated foam, or combinations thereof. If in foam form, the average pore size may vary according to needs of a prescribed therapy, for example, from about 400 microns to about 600 microns). Other physico-chemical properties of biodegradable components, such as tensile strength, may be chosen or manipulated, for example, to he suitable to needs of a prescribed treatment.

In some embodiments, particularly if biodegradable components are included, the dressing layer 110 may be characterized as having some biodegradable character or as exhibiting biodegradability. “Biodegradable” and “biodegradability” may individually or collectively refer to a characteristic of a material to disintegrate, degrade, or dissolve upon exposure to physiological fluids or processes, for example, when the dressing layer 110 is positioned with respect to a tissue site. For example, in some embodiments, the dressing layer 110 or a material from which the dressing layer 110 is formed may form a gel when contacted with an aqueous medium, such as water, saline, blood, or wound exudate. Such biodegradability may be exhibited as a result of chemical process or condition, a physical process or condition, or some combination thereof. For example, the biodegradable characteristics of the dressing layer 110 may substantially reduce or eliminate the need to remove the dressing layer 110 from a tissue site to which it is applied. In some embodiments, at least about 90% by weight of the biodegradable component (particularly at least about 95% by weight, at least about 99% by weight, or about 100% by weight) may be disintegrated, degraded, or dissolved with in a time period of from about 15 days to about 24 hours (particularly from about 12 days to about 36 hours or from about 10 days to about 48 hours), from introduction into a physiological environment when incubated with simulated physiological fluid at a temperature of about 37° C.

### Optional Dressing Layer Additives

In some embodiments, the dressing 100, and particularly the dressing layer 110, may optionally comprise one or more additional materials. Such optional components may include, for example, active materials such as preservatives, stabilizing agents, plasticizers, matrix strengthening materials, dyestuffs, and combinations thereof.

Additionally or alternatively, the dressing 100, and particularly the dressing layer 110, may comprise one or more additional active materials, for example, antimicrobial agents that may be effective to aid in wound healing. Non-limiting examples of such active materials may include non-steroidal anti-inflammatory drugs such as acetaminophen, steroids, anti-inflammatory cytokines, anaesthetics, antimicrobial agents such as penicillins or streptomycins, antiseptics such as chlorhexidine, growth factors such as a fibroblast growth factor (FGF), a platelet derived growth factor (PDGF), or an epidermal growth factor (EGF), and other well-known therapeutic agents, individually or in any combination. If present, such active materials may typically be included at any effective level that show therapeutic efficacy, while preferably not being at such a high level as to significantly counteract any critical or desired physical, chemical, or biological property of the dressing. Depending upon the therapeutic goal(s), the active material(s) may be loaded at a level of from about 10 wppm to about 10 wt % of the layer(s) in which it(they) is(are) present, for example, from about 50 wppm to about 5 wt % or from about 100 wppm to about 1 wt %.

In some embodiments, the antimicrobial agents may comprise a safe and effective amount of poly(hexamethylene biguanide) (“PHMB”), which is also known as polyaminopropyl biguanid (“PAPB”) and polyhexanide, having the following general formula.

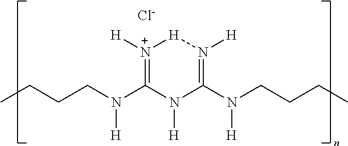

PHMB can be a cationic broad spectrum antimicrobial agent. PHMB may be synthesized by a variety of methods, including polycondensation of sodium dicyanamide and hexamethylenediamine. PHMB is commercially available from a variety of sources. In some embodiments, the PHMB may be present in one or more of the dressing layers at a level of from about 0.005 wt % to about 0.025 wt % of each layer in which it is present, particularly from about 0.007 wt % to about 0.2 wt % or from about 0.008 wt % to about 0.012 wt %, or in some cases at about 0.01 wt %. In some embodiments, the PHMB may be present in one or more of the dressing layers at a level of from about 0.05 wt % to about 3 wt % of each layer in which it is present, particularly from about 0.1 wt % to about 2.5 wt %, from about 0.3 wt % to about 2 wt %, from about 0.5 wt % to about 1.5 wt %, or in some cases at about 1 wt %. In alternative embodiments, silver compounds having antimicrobial efficacy may completely or partially replace the PHMB, as desired.

Additional or alternative antimicrobial agents can include, but are not necessarily limited to, silver, a silver salt, a tetracycline, a beta-lactam, a macrolide, an aminoglycoside, a fluoroquinolone, a cellulose ethyl sulfonate, or a combination thereof.

In some embodiments, the composition may comprise additional CMC as a modifier for one or more characteristics of the dressing or dressing layer(s), for example, the rheological, absorbency, and other structural characteristics. The additional CMC may be present in the layer(s) (other than the absorbent layer) at any level appropriate to result in the desired absorbency, rheological, or other structural characteristics of the dressing.

In some embodiments, the dressing layer 110 may contain a strengthening material, which can improve the handling characteristics of the dressing 100, for example, by decreasing its susceptibility to tearing. The strengthening material may comprise non-gelling cellulose fibers in some examples. Such non-gelling cellulose fibers may be substantially water insoluble and may be produced from cellulose that has not been chemically modified to increase water solubility, for example, as contrasted from carboxymethyl cellulose or other cellulose ethers. Non-gelling cellulose fibers are commercially available, such as under the tradename TENCEL (sold by Lenzing AG). In some embodiments, such fibers may be processed from a commercially-available continuous length, by cutting into lengths from about 0.5 to about 5 cm or from about 2 to about 3 cm in length. The non-gelling cellulose fibers may be present in the composition at any level appropriate to result in the desired physical characteristics of the composition. In general, when present, the non-gelling cellulose fibers may comprise from about 1% to about 55% of the layer by weight, particularly from about 5% to about 40% of the layer by weight or from about 10% to about 25% of the layer by weight. In some embodiments, if present, the non-gelling cellulose fibers can be characterized as additional or alternative reinforcing fibers and can be present in reinforcing fiber amounts.

### Dressing—Cover (Backing Layer)

In most embodiments, aside from dressing layer 110, the dressing 100 may comprise one or more additional layers. In various embodiments, such additional layers may perform any of a variety of functions including, for example, adherence of the dressing 100 to a tissue site or to surrounding tissues, increasing structural rigidity of the dressing, 100 protection from moisture or other contaminants in the external environment, protection of a wound surface, delivery of one or more active or other materials to the wound surface, or combinations thereof. In various embodiments, the additional layers may be conformable to a wound surface or to the surrounding tissues, for example, being capable of conforming such that the appropriate surfaces of the dressing 100 are in substantial contact with a tissue site.

For example, in the embodiment of FIG. 1, the dressing 100 comprises a backing layer 120, which may be positioned over the dressing layer 110, for example, so as to cover the dressing layer 110 at a tissue site. The backing layer 120 may have a first surface and a second surface. The backing layer 120 may support the dressing layer 110 on the second surface of the backing layer 120, for example, such that a first surface of the dressing layer 110 can be proximate to the second surface of the backing layer 120. In some embodiments, the first surface of the dressing layer may be in contact with and adhered to the second surface of the backing layer 120.

In particular embodiments, the backing layer 120 of the dressing 100 may extend beyond the boundaries or edges of the dressing layer 110, so as to exhibit an exposed backing layer margin, which may typically be exhibited on the second surface of the backing layer 120. In some embodiments, the backing layer 120 itself can be non-adherent.

In some embodiments, the backing layer 120 may generally be configured to provide a barrier to microbes, a barrier to external contamination, and protection from physical trauma. For example, the backing layer 120 may be constructed from a material that can reduce evaporative losses and provide a fluid seal between two components or two environments, such as between a therapeutic environment and a local external environment. The backing layer 120 may be formed from a suitable material, such as a polymer, for example, which may comprise or be an elastomeric film or membrane that can provide a seal at a tissue site. In some embodiments, the backing layer 120 may comprise or be a polyurethane. In some embodiments, the backing layer 120 may have a high moisture-vapor transmission rate (MVTR). For example, in such an embodiment, the MVTR may be at least 300 g/m² per twenty-four hours. For example, the backing layer 120 may comprise a polymer drape, such as a polyurethane film, that may be permeable to water vapor but generally impermeable to liquid water. In some embodiments, the backing or drape may have a thickness in the range of about from about 15 to about 50 microns.

Dressing—Secondary layer

Additionally, in some embodiments, the dressing 100 may further comprise a secondary layer, for example, positioned between the dressing layer 110 and the backing layer 120. In some embodiments, the secondary layer may comprise fluid pathways interconnected so as to improve distribution or collection of fluids. For example, in some embodiments, the secondary layer may be a porous material having a plurality of interconnected cells or pores. Suitable examples of porous material include a cellular foam such as an open-cell foam, a reticulated foam, or porous tissue collections. Other suitable porous material may include gauze or felted mat, which generally include pores, edges, or walls adapted to form interconnected fluid pathways. For example, in some embodiments, the secondary layer may be a foam having pore sizes in a range of 400-600 microns. In one non-limiting example, the secondary layer may be reticulated polyurethane foam.

In some embodiments having a secondary layer, the secondary layer may be characterized as exhibiting absorbency. For example, the secondary layer may exhibit an absorbency of at least 3 g saline/g, particularly at least 5 g saline/g, from 5 to 50 g saline/g, from 8 to 40 g saline/g, or from 8 to 20 g saline/g. In some embodiments, the secondary layer may be hydrophilic. In an example in which the secondary layer may be hydrophilic, the secondary layer may also wick fluid away from a dressing layer 110. In such embodiments, the wicking properties of the secondary layer may draw fluid away from dressing layer 110 by capillary flow or other wicking mechanisms. An example of a hydrophilic foam is a polyvinyl alcohol, open-cell foam. Other hydrophilic foams may include those made from or containing a polyether or a polyurethane. Additional or alternative foams that may exhibit hydrophilic characteristics include hydrophobic foams that have been treated or coated to provide hydrophilicity.

### Other Optional Dressing Layers

In some embodiments, the first surface of the dressing layer may be in contact with and adhered to the second surface of the backing layer 120. This adherence may, in some embodiments, result from an adherent layer disposed between the dressing layer 110 and the backing layer 120, thus constituting direct adherence. Such direct adherence means that the adherent layer, or at least the portion disposed between the dressing layer 110 and the backing layer 120, can be comprised of one or more different kinds of physical or chemical adhesive compositions. The adherent layer or portion thereof, in some embodiments, would not be expected to directly contact a tissue site. In embodiments with a backing layer margin extending beyond the dressing layer 110, the adherent layer may typically extend out to cover all or part of the backing layer margin. In such embodiments, the portion of the adherent layer on the margin may adhere the dressing 100 to a tissue site. Adherents that may directly contact tissue or that may be exposed to a treatment environment can typically have additional requirements, such as biocompatibility, and may be selected from a smaller list of physical or chemical adhesive compositions.

In some embodiments, such as where an adherent layer is an external layer in the dressing 100, for example to adhere the dressing 100 to a tissue site, the adherent layer can be releasably coupled to a release liner configured for removal before application to the tissue site, for example.

Adherence between the dressing layer 110 and the backing layer 120 may additionally or alternatively be indirect. For example, in some embodiments with a backing layer margin extending beyond the dressing layer 110, the adherent layer may be disposed on the backing layer margin and extend further over some portion of the dressing layer 110, such as the margin of the dressing layer 110. If this occurs without an adherent layer between the backing layer 120 and the dressing layer 110, the adherent layer may be said to indirectly adhere those layers, because those layers are each adhered to the adherent layer but not directly to each other. Such a configuration can allow an absorbent portion of the dressing layer 110 to expand differentially from the backing layer 120, for instance enabling relatively high levels of absorption of fluid with additional degrees of freedom. If the backing layer 120 is directly adhered to the dressing layer 110, the absorbent portion of the dressing layer 110 may expand into the tissue site, which can in some embodiments create undesirable pressure on a tissue site.

In particular embodiments, any adherent layer can comprise a hydrocolloid, a silicone adhesive, an acrylic adhesive, or a low melt fibrous adhesive precursor that can be heat-activated.

In some embodiments, absorbent material may be absent in or removed from a zone within the absorption layer. Such embodiments offer an additional or alternative mechanism enabling at least partial fluid absorptive expansion within the absorption layer, which can enable additional degrees of freedom for fluid absorption while creating no or little additional pressure on the wound site. For example, by creating a central zone in the absorbent layer of dressing layer 110 that is absent of material, the other portions of the absorbent layer can have extra volume to expand and can optionally experience increased fluid flow within the dressing layer 110, thus rendering the absorbent layer more efficient.

In some embodiments, the absorbent layer may be perforated to increase fluid flow, to reduce time to equilibrium absorption, or both. Such embodiments offer another additional or alternative mechanism enabling additional degrees of freedom for fluid absorption while creating no or little additional pressure on the wound site.

In some embodiments, the dressing 100 may include a contact layer, which may be non-adherent. For example, a contact layer may be disposed over the second surface of the dressing layer 110, opposite the backing layer 120. Non-adherent contact layers may be particularly advantageous in fibrinous situations to reduce potential adherence of the dressing layer 110 to a tissue site, to enable fluid to be effectively drawn away from the tissue site through the contact layer, or both. In some embodiments, therefore, the contact layer may be perforated, for example, for increased fluid flow. In various embodiments, the contact layer may comprise at least one of: an alkyl acrylate polymer, such as a methyl acrylate polymer, an ethyl acrylate polymer, or the like; an alkacrylate polymer, such as a methacrylate polymer, an ethacrylate polymer, or the like; and an alkyl alkacrylate polymer, such as a methyl methacrylate polymer, an ethyl methacrylate polymer, a methyl ethacrylate polymer, an ethyl ethacrylate polymer, or the like. Such (alk)acrylate polymers may be homopolymers but are more often copolymers, for example, with olefin comonomers. In particular, the non-adherent layer may comprise an ethylene-methyl acrylate copolymer, such as used in TIELLE™ Dressings and in SILVERCEL™ NON-ADHERENT Dressings available from Systagenix Wound Management, Limited. In various embodiments, the contact layer may comprise a silicone or polysiloxane polymer or copolymer. In such embodiments, the contact layer can extend over the absorbent layer, over the composite island, or over the dressing layer, as applicable, and over at least a portion of the backing layer margin. By extending over at least a portion of the backing layer margin, the portion of the contact layer extending over the backing layer margin may be coupled to the backing layer via the adherent layer. Optionally, the contact layer may have margin perforations, for example through which access can be allowed for the adherent layer to contact a wound site or tissue proximal thereto. Additionally or alternatively, particularly if the contact layer does not have sufficient tack to adhere the dressing to a tissue site, a second adherent layer may optionally be disposed on a surface of the contact layer opposite the backing layer, absorbent layer, composite island, or dressing layer.

### Negative-Pressure Therapy

Additionally, in some embodiments, a dressing layer such as the dressing layer 110, or a dressing comprising such a dressing layer, such as the dressing 100, may be employed in therapy, for example to treat a tissue site with reduced pressure. Treatment with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, micro-deformation of tissue at a wound site, and combinations thereof. Individually or together, these benefits may increase development of granulation tissue and reduce healing times.

In various embodiments, a negative-pressure therapy system may generally include a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a tissue site.

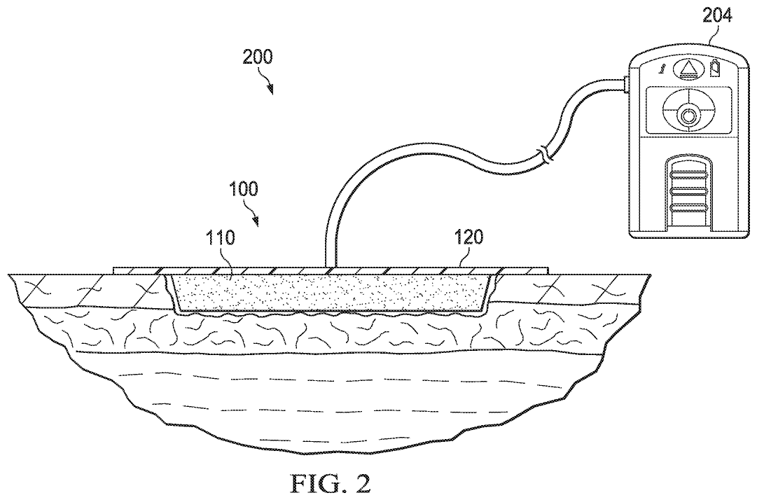

FIG. 2 is a simplified schematic of an example embodiment of a therapy system 200 that can provide negative-pressure therapy to a tissue site. In the example embodiment of FIG. 2, the dressing 100 is fluidly coupled to a negative-pressure source 204, such that negative pressure may be applied to a tissue site via the dressing 100.

For example, the dressing layer 110 may be generally configured to distribute negative pressure. For example, in some embodiments, the dressing layer 110 may comprise or be configured as a manifold. A “manifold” in this context generally includes any composition or structure providing a plurality of pathways configured to collect or distribute fluid across a tissue site under pressure. For example, a manifold may be configured to receive negative pressure from a negative-pressure source and to distribute negative pressure through multiple apertures or pores, which may have the effect of collecting fluid and drawing the fluid toward the negative-pressure source. More particularly, in the embodiment of FIG. 2, the dressing layer 110 is configured to receive negative pressure from the negative-pressure source 204 and to distribute negative pressure across the dressing layer 110. For example, this may have the effect of collecting fluid from a sealed space, such as by drawing fluid from the tissue site through the dressing layer 110. In additional or alternative embodiments, the fluid path(s) may be reversed or a secondary fluid path may be provided to facilitate movement of fluid across a tissue site. In some embodiments, the fluid pathways of a manifold may be interconnected to improve distribution or collection of fluids. In some embodiments, a manifold may comprise or be a porous foam material having a plurality of interconnected cells or pores. For example, open-cell foams generally include pores, edges, walls, or combinations thereof that may form interconnected fluid pathways, such as channels.

The fluid mechanics associated with using a negative-pressure source to reduce pressure in another component or location, such as within a sealed therapeutic environment, may be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, a fluid such as exudate flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications, such as by substituting a positive-pressure source for a negative-pressure source, and this descriptive convention should not be construed as a limiting convention.

As used herein, “negative pressure” is generally intended to refer to a pressure less than a local ambient pressure, such as the ambient pressure in a local environment external to a sealed therapeutic environment provided by the dressing 100. In many cases, the local ambient pressure may also be the atmospheric pressure proximate to or about a tissue site. Additionally or alternatively, the pressure may be less than a hydrostatic pressure associated with the tissue at the tissue site. Unless otherwise indicated, values of pressure stated herein are gauge pressures. Similarly, references to increases in negative pressure typically refer to a decrease in absolute pressure, for example a more negative pressure, while decreases in negative pressure typically refer to an increase in absolute pressure, for example a less negative pressure or a more positive pressure. While the amount and nature of negative pressure applied to a tissue site may vary according to therapeutic requirements, the pressure is generally a low vacuum, also commonly referred to as a rough vacuum, for example between −5 mm Hg (−667 Pa) and −500 trim Hg (−66.7 kPa). Common therapeutic ranges can be between −75 mm Hg (−9.9 kPa) and −300 mm Hg (−39.9 kPa).

In various embodiments, a negative-pressure supply, such as the negative-pressure source 204, may be a reservoir of air at a negative pressure. Alternatively, negative-pressure source 204 may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components that further facilitate therapy, such as sensors, processing units, alarm indicators, memory, databases, software, display devices, or user interfaces. For example, in some embodiments, the negative-pressure source 204 may be combined with a controller and other components into a therapy unit. A negative-pressure supply may have one or more supply ports configured to facilitate coupling and de-coupling of the negative-pressure supply to one or more distribution components.

In various embodiments, components may be fluidly coupled to each other to provide a path for transferring fluids between the components. For example, components may be fluidly coupled through a fluid conductor, such as a tube. The term “fluid conductor” is intended to broadly include a tube, pipe, hose, conduit, or other structure with one or more lumina adapted to convey a fluid between two ends thereof Typically, a fluid conductor may be an elongated, cylindrical structure with some flexibility, but the geometry and rigidity may vary. In some embodiments, the negative-pressure source 204 may be operatively coupled to the dressing 100 via a dressing interface. For example, in the embodiment of FIG. 2, the dressing 100 may be coupled to the negative-pressure source 204 via a dressing interface to receive negative pressure.

### Methods of Manufacture

Also disclosed herein are methods of manufacturing a dressing having an absorbent layer with elastic reinforcing fibers. In many embodiments, the method may comprise providing an elastic layer, such as a hydrocolloid or foam layer, having an original length and an absorbent layer comprising (i) from about 45% to about 90% carboxymethyl cellulose fibers and (ii) from about 10% to about 55% elastic reinforcing fibers. The elastic layer may then be stretched to an extended length that is at least 50% greater than the original, or relaxed, length. Thereafter, the fibers of the absorbent layer may be coupled, for example by lamination, to the extended elastic layer to form a composite laminate, so that only a portion of the fibers in the absorbent layer are coupled to the extended elastic layer, for example to allow for adherence during relaxation. In some embodiments, the lamination can occur using a layer comprising an adhesive, so as to couple the elastic layer to the layer comprising the adhesive and to couple the layer comprising the adhesive to the absorbent layer. In such embodiments, the layer comprising the adhesive can be deposited on the elastic layer, whether before, during, or after the extension of the elastic layer, to enable the coupling steps. Thereafter, the composite laminate may be relaxed to return the elastic layer to its original length. This relaxation step may advantageously cause bunching of the portion of the fibers in the absorbent layer that are not coupled to the elastic layer, while allowing the portion of the fibers in the absorbent layer that are coupled, albeit indirectly, to retain their coupling and advantageously to transfer some elastic character to the absorbent layer. In particular embodiments, the portion of the fibers in the absorbent layer coupled to the extended elastic layer may be sufficient for the composite laminate, the dressing, or both to exhibit a substantially elastic recovery under tissue treatment conditions. In some embodiments, the layer comprising the adhesive may include non-gelling fibers, and the coupling of the elastic layer to the layer comprising the adhesive and to couple the layer comprising the adhesive to the absorbent layer may occur approximately simultaneously, for instance by heating the non-gelling fibers to a temperature of at least about 10° C. below a melting point of the non-gelling fibers for about 1 minute to about 16 hours to establish effective coupling. In some such embodiments, the temperature can be from about 80° C. to about 150° C., for example from about 80° C. to about 130° C., from about 80° C. to about 120° C., from about 80° C. to about 110° C., from about 80° C. to about 100° C., from about 95° C. to about 150° C., from about 95° C. to about 130° C., from about 95° C. to about 120° C., from about 95° C. to about 110° C., from about 110° C. to about 150° C., from about 110° C. to about 130° C., or at about 120° C.

Alternatively, after providing an elastic layer, such as a hydrocolloid or foam layer, and an absorbent layer having an original length and comprising (i) from about 45% to about 90% carboxymethyl cellulose fibers and (ii) from about 10% to about 55% non-gelling (also non-woven) fibers, the fibers in the absorbent layer may be crimped or bunched up, such that the absorbent layer has an effective crimped length of at most 70% of the original, or relaxed, length. Thereafter, the crimped absorbent layer may be coupled, for example via lamination, to the elastic layer, such as in their crimped conformation, to form a composite laminate so that only a portion of the fibers in the absorbent layer are coupled to the elastic layer. In some embodiments, an adherent layer may be used as a coupling intermediate between the crimped absorbent layer and the elastic layer and may be disposed on the elastic layer before, during, or after crimping, as a step in the coupling process. By straining the composite laminate, and thus the absorbent layer, to un-bunch the crimping, for example in a length direction, the original, or relaxed, length of the absorbent layer can correspond to an extended length of the elastic layer. This straining or extension may be counteracted by the transference of elastic character through the coupling of the elastic layer and the absorbent layer. As a result, in particular embodiments, the portion of the fibers in the absorbent layer coupled to the extended elastic layer can be sufficient for the composite laminate, the dressing, or both to exhibit substantially elastic recovery under tissue treatment conditions. If a layer comprising an adhesive is used as a coupling intermediate, the layer comprising the adhesive may include non-gelling fibers in some embodiments, and the coupling of the elastic layer to the layer comprising the adhesive and to couple the layer comprising the adhesive to the absorbent layer may occur approximately simultaneously, for instance by heating the non-gelling fibers to a temperature of at least about 10° C. below a melting point of the non-gelling fibers for about 1 minute to about 16 hours to establish effective coupling. In some such embodiments, the temperature can be from about 80° C. to about 150° C., for example from about 80° C. to about 130° C., from about 80° C. to about 120° C., from about 80° C. to about 110° C., from about 80° C. to about 100° C., from about 95° C. to about 150° C., from about 95° C. to about 130° C., from about 95° C. to about 120° C., from about 95° C. to about 110° C., from about 110° C. to about 150° C., from about 110° C. to about 130° C., or at about 120° C.

### Methods of Use

Also disclosed herein are methods of treating a tissue site, for example, in the context of various therapies, such as eliminating, minimizing, or reducing edema, particularly in areas of relatively high articulation or flexure, such as during post-operative care. Non-limiting examples of areas implicating relatively high articulation or flexure include shoulder, elbow, knee, ankle, or hip joints, particularly knee or elbow joints.

In some embodiments, a therapy method may comprise positioning the dressing layer 110 with respect to a tissue site. For example, in operation, the dressing layer 110 may be positioned proximate to the tissue site. The dressing layer 110 may be used with any of a variety of wounds, such as those occurring from trauma, surgery, or disease. For example, the dressing layer 110 may be placed within, over, on, or otherwise proximate to the tissue site. Additionally, in some embodiments, a cover such as the backing layer 120 may be placed over the dressing layer 110 and sealed to an attachment surface near the tissue site. For example, the backing layer 120 may be sealed to undamaged epidermis peripheral to a tissue site. In some embodiments. the dressing layer 110 may be positioned and the backing layer 120 may be positioned thereafter. In some embodiments, the dressing layer 110 and backing layer 120 may be preassembled, for example, such that the dressing layer 110 and backing layer 120 are positioned with respect to each other prior to placement proximate the tissue site. Thus, the backing layer 120 can provide a sealed therapeutic environment including the dressing layer 110 and proximate to a tissue site, substantially isolated from the external environment.

Additionally, in some negative-pressure therapy embodiments, a negative-pressure therapy may comprise positioning the dressing layer 110 and backing layer 120 proximate to a tissue site. For example, the various components of the dressing layer 110 may be positioned with respect to the tissue site sequentially or, alternatively, may be positioned with respect to each other and then positioned with respect to the tissue site. The negative-pressure therapy may further comprise sealing the dressing layer 110 to tissue surrounding the tissue site to form a sealed space. For example, the backing layer 120 may be placed over the dressing layer 110 and sealed to an attachment surface near the tissue site, such as undamaged epidermis peripheral to a tissue site. Thus, the dressing layer 110 and backing layer 120 can provide a sealed therapeutic environment proximate to the tissue site, substantially isolated from the external environment.

The negative-pressure therapy method may further comprise fluidly coupling a negative-pressure source to the sealed space and operating the negative-pressure source to generate a negative pressure in the sealed space. For example, the negative-pressure source 204 may be coupled to the dressing 100 such that the negative-pressure source 204 may be used to reduce the pressure in the sealed space. For example, negative pressure applied across the tissue site via the dressing 100 may be effective to induce macrostrain and microstrain at the tissue site, as well as to remove exudates and other fluids from the tissue site.

### Embodiments

Additional or alternative examples may include one or more of the following embodiments.

Embodiment 1. A method of manufacturing a dressing having an absorbent layer with elastic reinforcing fibers, the method comprising: providing an elastic layer having an original length and an absorbent layer comprising (i) from about 45% to about 90% carboxymethyl cellulose fibers and (ii) from about 10% to about 55% reinforcing fibers; extending the elastic layer to an extended length at least 50% greater than the original length; before, after, or during the extending step, depositing a layer comprising an adhesive on the elastic layer; coupling the layer comprising the adhesive to the elastic layer; coupling the fibers of the absorbent layer to the layer comprising the adhesive to form a composite laminate, so that only a portion of the fibers in the absorbent layer are coupled to the extended elastic layer via the layer comprising the adhesive; and relaxing the composite laminate to return the elastic layer to its original length, thereby causing bunching in a portion of the fibers in the absorbent layer that are not coupled to the extended elastic layer.

Embodiment 2. The method of embodiment 1, wherein the elastic layer comprises a hydrocolloid or a foam.

Embodiment 3. The method of embodiment 1 or embodiment 2, wherein the portion of the fibers in the absorbent layer coupled to the extended elastic layer are sufficient for the composite laminate, the dressing, or both to exhibit a substantially elastic recovery under tissue treatment conditions.

Embodiment 4. The method of any of embodiments 1-3, wherein the layer comprising the adhesive comprises non-gelling fibers, wherein the two coupling steps occur approximately simultaneously when the non-gelling fibers are heated to a temperature of at least about 10° C. below a melting point of the non-gelling fibers for about 1 minute to about 16 hours to establish effective coupling.

Embodiment 5. A dressing for incision management comprising a dressing layer that comprises: an elastic layer; and an absorbent layer coupled, for example by lamination, to the elastic layer and which comprises: (i) from about 45% to about 90% carboxymethyl cellulose fibers; and (ii) from about 10% to about 55% elastic reinforcing fibers.

Embodiment 6. The dressing of embodiment 5, wherein the elastic layer comprises a hydrocolloid or a foam, and wherein the dressing a width and a length perpendicular to the width and exhibits a substantially elastic response when exposed to about 10% strain along the length for about 10 minutes.

Embodiment 7. The dressing of embodiment 5 or embodiment 6, further comprising a non-adherent backing layer disposed on the absorbent layer on a surface opposite the elastic layer.

Embodiment 8. The dressing of embodiment 7, wherein the non-adhesive backing layer comprises a polyurethane.

Embodiment 8. The dressing of embodiment 6 or embodiment 7, further comprising a non-adherent layer disposed on the elastic layer on a surface opposite the absorbent layer.

Embodiment 9. The dressing of embodiment 8, wherein the non-adherent layer is perforated and comprises at least one of an alkyl acrylate polymer, an alkacrylate polymer, and an alkyl alkacrylate polymer.

Embodiment 10. The dressing of embodiment 9, wherein the alkyl acrylate polymer comprises an ethylene-methyl acrylate copolymer.

Embodiment 11. The dressing of any of embodiments 4-10, further comprising a wound healing agent in or on the elastic layer, the absorbent layer, the non-adherent layer, if present, or a combination thereof.

Embodiment 12. The dressing of embodiment 11, wherein the wound healing agent comprises a non-steroidal anti-inflammatory drug, a steroid, an anti-inflammatory cytokine, an anaesthetic, an antiseptic, an antimicrobial agent, a growth factor, or a combination thereof.

Embodiment 13. The dressing of embodiment 12, wherein the growth factor comprises a platelet-derived growth factor (PDGF), a fibroblast growth factor (FGF), an epidermal growth factor (EGF), or a combination thereof.

Embodiment 14. The dressing of embodiment 13, wherein the antimicrobial agent comprises silver, a silver salt, a tetracycline, a beta-lactam, a macrolide, an aminoglycoside, a fluoroquinolone, a cellulose ethyl sulfonate, or a combination thereof.

Embodiment 15. The dressing of any of embodiments 4-7 and 11-14, further comprising an adherent layer disposed on the elastic layer on a surface opposite the absorbent layer and a release liner disposed on the adherent layer on a surface opposite the elastic layer.

Embodiment 16. The dressing of any of embodiments 4-15, wherein the elastic reinforcing fibers comprise polymers or copolymers of olefins or amides.

Embodiment 17. The dressing of any of embodiments 4-16, wherein the absorbent fibers are laminated to the elastic layer.

Embodiment 18. The dressing of embodiment 17, wherein a layer of non-gelling fibers is disposed between the absorbent layer and the elastic layer, and wherein the non-gelling fibers are heated to a temperature of at least about 10° C. below a melting point of the non-gelling fibers for about 1 minute to about 16 hours to establish effective lamination.

Embodiment 19. The dressing of embodiment 18, wherein the non-gelling fibers are deposited as an at least partially inter-connected scrim.

Embodiment 20. A method of reducing edema for a wound site surrounded by tissue, the method comprising positioning a dressing made according to the method of any of embodiments 1-4 or a dressing according to any of embodiments 5-19 near the wound site and releasably adhering the dressing to the tissue.

Embodiment 21. The method of embodiment 20, wherein the wound site is proximal to a knee joint or an elbow joint.

Embodiment 22. The method of embodiment 20 or embodiment 21, further comprising: sealing the dressing within a sealed space adjacent to the wound site; and applying a negative pressure to the sealed space.

### Non-Limiting Discussion of Terminology

The description and specific examples above are provided for illustration only and are not intended to limit the scope of the claimed subject matter. Moreover, recitation of multiple embodiments having stated features does not exclude other embodiments having additional features, or other embodiments incorporating different combinations of the stated features. Components may also be combined or eliminated in various configurations for purposes of sale, manufacture, assembly, or use. Specific examples are provided for illustrating how to make and use the compositions, and examples of methods are not intended to be a representation that given embodiments have, or have not, been made or tested. Equivalent changes, modifications and variations of some embodiments, materials, compositions and methods can be made within the scope of the appended claims, with substantially similar results.

As used herein, the words “include,” “contain,” and their variants, arc intended to be non-limiting, such that recitation of items in a list is not necessarily to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments that do not contain those elements or features. Moreover, descriptions of various alternatives using terms such as “or” do not require mutual exclusivity unless clearly required by the context, and the indefinite articles “a” or “an” do not limit the subject to a single instance unless clearly required by the context.

Although the open-ended term “comprising,” as a synonym of non-restrictive terms such as including, containing, or having, is used herein to describe example embodiments, embodiments may alternatively be described using more limiting terms such as “consisting of” or “consisting essentially of.” Thus, for any given embodiment reciting materials, components or process steps with such open-ended terms, similar or analogous embodiments are contemplated consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosure of values and ranges of values for specific parameters, such as temperatures, molecular weights, weight percentages, etc., are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, disclosure of two or more ranges of values for a parameter, whether such ranges are nested, overlapping or distinct, may subsume all possible combination of ranges for the value that might be claimed using endpoints of the disclosed ranges. For example, if parameter X is exemplified herein to have values in the range of 1-10, or 2-9, or 3-8, Parameter X may be envisioned as having other ranges of values including 1-2, 1-3, 1-8, 1-9, 2-3, 2-8, 2-10, 3-9, 3-10, 8-9, 8-10, and 9-10.

The term “about,” as used herein, is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and other deviations as will be apparent by those of skill in the art from the context of this disclosure. For example, unless otherwise defined by the specification per se or by the context of the specification, the term “about,” with reference to a value, may refer to any number that would round to that value, based on a significant digit analysis. In such a circumstance, a value of “about 30%”, assuming the “3” is the only significant digit, could encompass from 25% to just below 35%. However, the context of the specification would limit that interpretation based on significant digits, so that the “about” ranges do not overlap. For example, if the specification discloses ranges that include “about 25%, about 30%, about 35%,” etc., about 30% in that context could encompass from 27.5% to just below 32.5%. Alternatively, the term “about” may refer to deviations that are greater or lesser than a stated value or range by ±10% of the stated value(s), as appropriate from the context of the disclosure. In such a circumstance, a value of “about 30%” may encompass from 27% to 33%. Whether or not modified by the term “about,” quantitative values recited herein include equivalents to the recited values, for example, deviations from the numerical quantity, as would be recognized as equivalent by a person skilled in the art in view of this disclosure.

The appended claims set forth novel and inventive aspects of the subject matter disclosed and described above, but the claims may also encompass additional subject matter not specifically recited in detail. For example, certain features, elements, or aspects may be omitted from the disclosure and claims, if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention, as defined by the appended claims.

1. A method of manufacturing a dressing having an absorbent layer with elastic reinforcing fibers, the method comprising:
providing an elastic layer having an original length and an absorbent layer comprising (i) from about 45% to about 90% carboxymethyl cellulose fibers and (ii) from about 10% to about 55% reinforcing fibers;
extending the elastic layer to an extended length at least 50% greater than the original length;
before, after, or during the extending step, depositing a layer comprising an adhesive on the elastic layer;
coupling the layer comprising the adhesive to the elastic layer;
coupling the fibers of the absorbent layer to the layer comprising the adhesive to form a composite laminate, so that only a portion of the fibers in the absorbent layer are coupled to the extended elastic layer via the layer comprising the adhesive; and
relaxing the composite laminate to return the elastic layer to its original length, thereby causing bunching in a portion of the fibers in the absorbent layer that are not coupled to the extended elastic layer.

2. The method of claim 1, wherein the elastic layer comprises a hydrocolloid or a foam.

3. The method of claim 1, wherein the portion of the fibers in the absorbent layer coupled to the extended elastic layer are sufficient for the composite laminate, the dressing, or both to exhibit a substantially elastic recovery under tissue treatment conditions.

4. The method of claim 1, wherein the layer comprising the adhesive comprises non-gelling fibers, wherein the two coupling steps occur approximately simultaneously when the non-gelling fibers are heated to a temperature of at least about 10° C. below a melting point of the non-gelling fibers for about 1 minute to about 16 hours to establish effective coupling.

5. A dressing for incision management comprising:
an elastic layer; and
an absorbent layer coupled to the elastic layer and comprising:
(i) from about 45% to about 90% carboxymethyl cellulose fibers; and
(ii) from about 10% to about 55% elastic reinforcing fibers.

6. The dressing of claim 5, wherein the elastic layer comprises a hydrocolloid or a foam, and wherein the dressing a width and a length perpendicular to the width and exhibits a substantially elastic response when exposed to about 10% strain along the length for about 10 minutes.

7. The dressing of claim 5, further comprising a non-adhesive backing layer disposed on the absorbent layer on a surface opposite the elastic layer.

8. The dressing of claim 7, wherein the non-adhesive backing layer comprises a polyurethane.

9. The dressing of claim 7, further comprising a non-adherent layer disposed on the elastic layer on a surface opposite the absorbent layer.

10. The dressing of claim 9, wherein the non-adherent layer is perforated and comprises an ethylene-methacrylate copolymer.

11. The dressing of claim 10, further comprising a wound healing agent in or on the elastic layer, the absorbent layer, the non-adherent layer, if present, or a combination thereof.

12. The dressing of claim 11, wherein the wound healing agent comprises a non-steroidal anti-inflammatory drug, a steroid, an anti-inflammatory cytokine, an anaesthetic, an antiseptic, an antimicrobial agent, a growth factor, or a combination thereof.

13. The dressing of claim 12, wherein the growth factor comprises a platelet-derived growth factor (PDGF), a fibroblast growth factor (FGF), an epidermal growth factor (EGF), or a combination thereof.

14. The dressing of claim 12, wherein the antimicrobial agent comprises silver, a silver salt, a tetracycline, a beta-lactam, a macrolide, an aminoglycoside, a fluoroquinolone, a cellulose ethyl sulfonate, or a combination thereof.

15. The dressing of claim 14, further comprising an adherent layer disposed on the elastic layer on a surface opposite the absorbent layer and a release liner disposed on the adherent layer on a surface opposite the elastic layer.

16. The dressing of claim 15, wherein the elastic reinforcing fibers comprise polymers or copolymers of olefins or amides.

17. The dressing of claim 16, wherein the absorbent fibers are laminated to the elastic layer.

18. The dressing of claim 17, wherein a layer of non-gelling fibers is disposed between the absorbent layer and the elastic layer, and wherein the non-gelling fibers are heated to a temperature of at least about 10° C. below a melting point of the non-gelling fibers for about 1 minute to about 16 hours to establish effective lamination.

19. The dressing of claim 18, wherein the non-gelling fibers are deposited as an at least partially inter-connected scrim.

20. A method of reducing edema for a wound site surrounded by tissue, the method comprising:
providing a dressing having an elastic layer, and an absorbent layer coupled to the elastic layer and comprising from about 45% to about 90% carboxymethyl cellulose fibers, and from about 10% to about 55% elastic reinforcing fibers; and
positioning the dressing near the wound site and releasably adhering the dressing to the tissue.

21. The method of claim 20, wherein the wound site is proximal to a knee joint or an elbow joint.

22. The method of claim 20, further comprising:
sealing the dressing within a sealed space adjacent to the wound site; and
applying a negative pressure to the sealed space.

23. (canceled)
